(19) United States
(12) Patent Application Publication
(10) Pub. No.: US 2002/0048587 A1
(43) Pub. Date: Apr. 25, 2002

(54) Sugar ethers as adjuvants in immunization compositions

(76) Inventors: Jerome Aucouturier, Chatenay Malabry (FR); Sebastien Deville, Paris (FR); Laurent Dupuis, Castres (FR); Gerard Trouve, Casters (FR); Vincent Ganne, La Varenne Saint Hilaire (FR)

Correspondence Address: Robert M. Schulman, Esq., Hunton & Williams, Suite 1200, 1900 K Street, N.W., Washington, DC, 20006, US

(21) Appl. No.: 09/877,215

(22) Filed: Jun. 11, 2001

(30) Foreign Application Priority Data
Jun. 9, 2000 (FR) 0007408

(51) Int. Cl. A61K 39/00

(52) U.S. Cl. 424/184.1; 424/185.1

(57) ABSTRACT

Use of ethers of sugars, as adjuvants of immunity in immunization compositions, therapeutic compositions containing them and the use thereof as vaccines

Use of a mixture comprising a compound of formula (I):

R₁—O-(G) x-H (I)

in which R₁ represents a saturated or unsaturated, linear or branched hydrocarbon-based radical comprising

1 to 30 carbon atoms, G represents the residue of a saccharine and x represents a decimal number between 1 and 5, or a mixture of compounds of formula (I) and, if desired, a compound of formula (II):

R₁—OH (II)

in which R2 represents, independently of R₁, a saturated or unsaturated, linear or branched hydrocarbon-based radical comprising from 8 to 30 carbon atoms, or a mixture of compounds of formula (II), as an adjuvant of immunity in an immunization composition comprising at least one antigen or at least one in vivo generator of a compound comprising an amino acid sequence, and immunization composition comprising said compounds of formulae (I) and (II).

### FIELD OF THE INVENTION

The present invention relates to novel adjuvants for immunization compositions, and also to compositions comprising at least one antigen, in particular an antigen of viral, bacterial or parasitic origin, and at least one adjuvant.

### BACKGROUND OF THE INVENTION

No There is an ever-increasing development of inactivated vaccines or vaccines containing purified antigens, since such a development makes it possible to avoid undesirable side effects However, the improvement in the quality of the antigens occurs to the detriment of their immunogenic nature. For this reason, they are combined with adjuvants of immunity.

Adjuvants of immunity are products which increase the reactions of the immune system, when they are administered in the presence of antigens of viral, bacterial or parasitic origin. They cause the massive appearance of macrophages at the site of injection, and then in the lymph nodes, increase the production of specific immunoglobulins, antibodies, and stimulate many cells involved in the mechanisms of the immune defense.

These adjuvants are diverge in nature. They may, for example, consist of mixtures of oils and of surfactants producing vaccines in the form of lyposomes or of emulsions.

Freund's adjuvants are very effective; they result from the combination of a mineral oil and of a mannitol ester, which may or may not contain a killed mycobacterium.

Vaccines prepared by mixing, in equal parts, a Freund's adjuvant and an aqueous antigenic medium are still the references throughout the world for laboratory studies. They are in the form of water-in-oil (W/O) emulsions, i.e., of emulsions in which the continuous phase is oil. Now, these emulsions are very viscous and are therefore difficult to inject. They are also relatively unstable, since phase separations are observed only a few days after they have been prepared.

As conventional adjuvants, there are also metal salts, such as aluminum hydroxide, cerium nitrate, zinc sulphate, colloidal iron hydroxide or calcium chloride. Among them, aluminum hydroxide is the most commonly used. These adjuvants are described in the article by Rajesh K. Gupta et al. “Adjuvants, balance between toxicity and adjuvanticity”, Vaccine, Vol.11, Issue 3,1993, pages 293-306. However, their immunostimulating effectiveness is poor and they sometimes induce, when these therapeutic compositions are injected, the formation of lesions and other local reactions, such as granulomes, at the point of injection.

More recently, it has been discovered that water-soluble salts of divalent or trivalent metals are good adjuvants of immunity. Among them, manganese gluconate, calcium gluconate, manganese glycerophosphate, soluble aluminum acetate or aluminum salicylate are the most promising. Such adjuvants are described in the international patent applications published under the numbers WO 96/32964 and WO 98/17311.

As other adjuvants of immunity, in particular in the case of mucosal administration, mention may be made of sympathomimetic compounds described in the international patent application published under the number WO 98/15288. While investigating the development of novel adjuvants, the applicant has discovered that certain surfactants. themselves, are effective as immuno-stimulants in the absence of oils, and that it is thus possible to prepare aqueous immunization compositions comprising one or more of these agents as immuno-stimulants.

### SUMMARY OF THE INVENTION

The present invention provides a mixture comprising:

a) a compound of formula (I):

R₁—O-(G)x-H (I)

in which R˜ represents a saturated or unsaturated, linear or branched hydrocarbon-based radical comprising 1 to 30 carbon atoms, G represents the residue of a saccharine and x represents a decimal number between 1 and 5, or a mixture of compounds of formula (I) and/or

b) a compound of formula (II):

R₂—OH (II)

in which R₂ represents, independently of R₁ a saturated or unsaturated, linear or branched hydrocarbon-based radical comprising from 8 to 30 carbon atoms, or a mixture of compounds of formula (II) as an adjuvant of immunity in an immunization composition comprising at least one antigen or at least one in vivo generator of a compound comprising an amino acid sequence; and methods of using same, and preparing same.

### DETAILED DESCRIPTION OF THE PREFERRED EMBODIMENTS

The expression “residue of a saccharine” is intended to refer to, for G, a bivalent radical resulting from the removal, from a sugar molecule, on the one hand, of a hydrogen atom of one of its hydroxyl groups and, on the other hand, the anomeric hydroxyl group. The term “saccharine” refers in particular to glucose or dextrose, fructose, mannose, galactose, altrose, idose, arabinose, xylose, ribose. gulose, lyxose, maltose, matotriose, lactose. cellobiose, dextran, talose, allose, raftinose, levoglucan, cellulose or starch. The oligomeric structure (G)ₓ may exist in any form of isomerism, whether it is optical isomerism, geometric isomerism or positional isomerism, it may also represent a mixture of isomers.

The number x, which represents, in formula (I) the mean degree of polymerization of the saccharine, is more particularly between 1 and 3, in particular between 1.05 and 2.5, most particularly between 1.1 and 2.0, and preferably less than or equal to 1.5.

G represents more particularly the residue of glucose or the residue of xylose.

The R₁ radical represents in particular a radical comprising from 5 to 22 carbon atoms, chosen from pentyl. hexyl, heptyl, octyl, nonyl, decyl, undecyl, dodecyl, tridecyl, tetradecyl, pentadecyl, hexadecyl, heptadecyl, octadecyl, nonadecyl, eicosyl, uneicosyl, docosyi, hentadecenyl, eicosenyl, uneicosenyl, docosenyl or heptadecadienyl or decenyl radicals, said radicals being linear or branched.

R₂ represents more particularly a radical comprising from 8 to 22 carbon atoms, chosen from octyl. nonyl. decyl, undecyl. dodecyl, tridecyl. tetradecyl. pentadecyl, hexadecyl, heptadecyl, octadecy nonadecyl, eicosyl, uneicosyl, docosyl, heptadecenyl, eicosenyl. uneicosenyl, docosenyl or hepradecadienyl or decenyl radicals, said radicals being linear or branched.

When the mixture as defined above comprises at least one compound of formula (I) and at least one compound of formula (II), the compound of formula (I)/compound of formula (II) weight ratio is generally between 10/90 and 90/10, more particularly between 10/90 and 60/40.

The expression “antigen or at least one in vivo generator of a compound comprising an amino acid sequence” is intended to refer to either killed micro-organisms, such as viruses, bacteria or parasites, purified fractions of these micro-organisms, or living micro-organisms, the pathogenic power of which has been attenuated. By way of viruses which may constitute an antigen according to the present invention, mention may be made of the rabies virus, herpes viruses, such as Aujeszky's disease virus, orthomixoviruses such as influenzae virus, picornaviruses such as foot-and-mouth virus, or retroviruses such as HIV. By way of a micro-organism of the bacterial type which may constitute an antigen according to the invention, mention may be made of E. Coli, and those of the Pasteurella, Furunculosis. Vibrio, Staphylococcus and Streptococcus genera. By way of a parasite, mention may be made of those of the Tryoanosoma, Plasmodiun and Leishniania genera. Mention may also be made of recombinant viruses, in particular nonenveloped viruses such as adenoviruses. the vaccinia virus, the canarypox virus. herpesviruses or baculoviruses. Reference is also made to a recombinant living nonenveloped viral vector, the genome of which contains, preferably inserted into a portion which is not essential for the replication of the corresponding enveloped virus, a sequence encoding an antigenic subunit which induces antibody synthesis and/or a protective effect against the above-mentioned enveloped virus or pathogenic micro-organism. These antigenic subunits may, for example. be a protein, a glycoprotein, a peptide or a fraction which is peptide in nature and/or which is protective against infection with a living micro-organism such as an enveloped virus, a bacterium or a parasite. The exogenist gene inserted into the micro-organism may, for example, be derived from an Aujeszky or HIV virus.

Mention may in particular be made of a recombinant plasmid consisting of a nucloetide sequence into which an exogenous nucloetide sequence, originating from a pathogenic virus or micro-organism, is inserted. The purpose of the latter nucleotide sequence is to allow the expression of a compound comprising an amino acid sequence, the purpose of the compound, itself, being to trigger an immune reaction in a host organism.

The expression ‘in vivo’ generator of a compound comprising an amino acid sequence'is intended to refer to any biological product capable of expressing said compound in the host organism into which said in vivo generator has been introduced. The compound comprising the amino acid sequence can be .a protein, a peptide or a glycoproteirn. These in vivo generators are generally obtained using processes derived from genetic engineering. More particularly, they may consist of living micro-organisms, generally a virus, playing the role of a recombinant vector, into which a nucleotide sequence, in particular an exogenous gene, is inserted. These compounds are known in themselves, and are used in particular as recombinant subunit vaccines. In this respect, reference may be made to the article by M. Eloit et al., Journal of Virology (1990) 71, 2925-2431 and to the international patent applications published under the numbers WO-A-91/00107 and WO-A-94/l6681. The in vivo generators according to the invention may also consist of a recombinant plasmid comprising an exogenous nucleotide sequence, capable of expressing, in a host organism, a compound comprising an amino acid sequence. Such recombinant plasmids and their method of administration in a host organism were described in 1990, by LIN et al., Circulation 82: 2217. 2221: Cox et al., J. of Virol, September 1993, 67. 9, 5564-5567 and in the international application published under the number WO 95/25542. Depending on the nature of the nucleotide sequence included in the in vivo generator, the compound comprising the amino acid sequence, which is expressed in the host organism, may:

(i) be an antigen and allow the triggering of an immune reaction,

(ii) have a curative action with respect to a disease, essentially a disease which is functional in nature, which has been triggered in the host organism. In this case, the in vivo generator allows host treatment of the gene therapy type.

By way of example, such a curative action may consist of synthesis, by the in vivo generator, of cytokines. such as interleukins, in particular interleukin 2. The latter allow the triggering or reinforcement of an immune reaction directed towards the selective elimination of cancer cells.

According to a first particular aspect of the present invention, a subject of the latter is the use as defined above, in an immunization composition comprising an aqueous phase and one or more oils chosen from mineral, animal or plant oils, in a weight proportion in oily phase of between 10 and 90% of the total weight thereof, and preferably between 30% and 70% by weight of the total weight thereof.

When the immunization composition comprises an aqueous phase and an oily phase, it is generally in the form of a water-in-oil (W/O) emulsion, of an oil-in-water (O/W) emulsion, of a water-in-oil-in-water (W/O/W) emulsion or of a microemulsion.

According to a second particular aspect of the present invention, a subject of the latter is the use of a mixture comprising:

a) a compound of the formula (I):

R₁—O-(G)x-H (I)

as defined above, or a mixture of compounds of formula (I), and, if desired,

b) a compound of formula (II)

R₂—OH (II)

as defined above, or a mixture of compounds of formula (II). as an adjuvant of immunity in an immunization composition comprising an aqueous phase and not comprising an oily phase.

A subject of the invention is also a composition in the form of an aqueous phase comprising:

(i) at least one antigen or at least one in vivo generator of a compound comprising an amino acid sequence and,

(ii) at least one compound of formula (I)

R₁—O-(G)x-H (I)

and, if desired,

(iii) at least one compound of formula (II), for which formulae (I) and (II) have the meanings indicated above.

When the composition as defined above comprises at least one compound of formula (I) and at least one compound of formula (II), the compound of formula (I)/compound of formula (II) weight ratio is generally between 10/90 and 90/10, more particularly between 10/90 and 60/40.

The composition as defined above may also comprise one or more oils chosen from mineral, animal or plant oils, in a weight proportion in oily phase of between 10 and 90% of the total weight thereof, and preferably between 30% and 70% by weight of the total weight thereof.

When the composition comprises an aqueous phase and an oily phase, it is generally in the form of a water-in-oil (W/O) emulsion, of an oil-in-water (O/W) emulsion, of a water-in-oil-in-water (W/O/W) emulsion or of a microemulsion.

A composition as defined above may also comprise an aqueous phase and not comprise an oily phase.

A composition according to the invention comprises a concentration of antigen which depends on the nature of this antigen and on the nature of the individual treated. It is, however, particularly noteworthy that an adjuvant according to the invention makes it possible to notably decrease the conventional antigen dose required. The suitable concentration of antigen can be determined conventionally by those skilled in the art. Generally, this dose is about 0.1 μg/cm³ to 1 g/cm³, more generally between 1 μg/cm³ and 100 mg/cm³.

The concentration of said in vivo generator in the composition according to the invention depends, once again, in particular on the nature of said generator and of the host to which it is administered. This concentration can be easily determined by those skilled in the art, on the basis of routine experiment. By way of indication, it may, however, be specified that, when the in vivo generator is a recombinant micro-organism, the concentration thereof in the composition according to the invention can be between 10² and 10¹⁵ micro-organisms/cm³, preferably between 10⁵ and 10² micro-organisms/cm³. When the in vivo generator is a recombinant plasmid, the concentration thereof in the composition is between 0.01 mg and 100 mg/cm³.

A composition which is the subject of the present invention contains between 0.2 mg/cm³ and 500 mg/cm³ of adjuvant, more particularly between 2 mg/cm³ and 500 mg/cm³ of adjuvant, and preferably between 50 mg/cm³ and 200 mg/cm³ of adjuvant.

The compounds of formulae (I) and (II) are preferably pharmaceutically acceptable and well tolerated by the human or animal organism; they must, in particular, be free of heavy metals and have very low acid or peroxide indices. It is also desirable for them to satisfy the standards determined by innocuity tests, in particular those described by S. S. Berilin, Annals of Allergy. 1962, 20, 473, or those described in the European Pharmacopoeia.

The composition according to the invention can also comprise a conventional immunostimulant such as Avridine™, N,N-dioctadecyl-N′N′-bis(2-hydroxyethyl)-propanediamine, MDP (muramyl dipeptide) derivatives, in particular threonyl-MDP, mycolic acid or Lipid A derivatives.

The composition according to the invention can also comprise one or more water-soluble metal cation organic salts, such as for example calcium gluconate, manganese gluconate, manganese glycerophosphate, calcium glycerophosphate, calcium fructoheptonate, manganese fructoheptanate. aluminum salicylate or soluble aluminum acetate.

When the adjuvant composition according to the invention comprises one or more salts, the latter is at a concentration of 0.02 to 3000 mg/cm³, preferably 0.1 mg to 1000 mg/cm³, more preferably from 0.1 mg to 150 mg/cm³. The composition according to the invention can comprise a sympathomimetic compound.

The term “sympathnmimetic compounds” is intended to refer in particular to amphetamines, catecholamines, phenylisoproylamines or tyramine. As examples of such compounds, mention may be made in particular of isoproterenol, L-adrenaline, levarterenol, ephedrine, phenylephedrine, or salbutamol.

When the adjuvant composition according to the invention comprises a symnathomimetic compound, the latter is at a concentration of 10⁻¹⁰ molar to 10⁻² molar, preferably of 10⁻⁷ molar to 10⁻⁵ molar.

The composition according to the invention can be used as a preventive or curative medicinal product. Depending on the nature of the antigen or of the in vivo generator, a composition according to the invention can be administered to fish, crustaceans such as shrimp, poultry, in particular geese, turkeys. pigeons and chickens, to Canidae such as dogs, to Felidae such as cats, to pigs, to primates, to Bovidae, to Ovidae and to horses. The composition according to the invention can also be administered to humans. The administration of the composition can be carried out conventionally via the parenteral route, in particular by subcutaneous, intramuscular or intraperitoneal injection, or via the mucosal route, in particular orally, rectally, nasally or vaginally. According to another aspect of the invention, the latter consists of the use of an adjuvant as defined above, for preparing a vaccine intended for preventing or for treating an infectious disease, in particular an infectious disease engendered by a virus or a micro-organism such as those mentioned above.

According to another final aspect of the invention, the latter consists of the use of this adjuvant for preparing a composition intended to treat a disease which is functional in nature, such as cancer or cystic fibrosis.

The following examples illustrate the invention without limiting it, however.

A) Surfactants used

The surfactants used are as follows:

| Composition % | Alkylpolyglucosides | Alcohols of the | |
|---|---|---|---|
| weight | of formula (I) | formula (II) | Water |
| Composition | = 12% C₁₆ and C₁₈ R₁ | = 87% C₁₆ and C₁₈ R₂ | 0% |
| (A) | | | |
| Composition B | = 14.5% C₁₂ + R₁ | = 1.5% C₁₄ + R₂ | 0% |
| | = 19% C₁₄ + R₁ | = 4.5 C₁₈ + R₂ | |
| | = 11% C₁₆ + R₁ | = 28% C₁₈ R₂ | |
| | = 21.5% C₁₈ R₁ | | |
| Composition C | 50-55% C₁₀-C₁₄ R₁ | <5% | 40-45% |
| Composition D | = 14.5% isostearyl R₁ | = 85.5% isostearyl R₂ | 0% |
| Composition E | = 11% isostearyl R₁ | = 90% isostearyl R₂ | 0% |
| Composition F | = 16.5% oleyl R₁ | = 83.5% oleyl R₂ | 0% |

### EXAMPLE 1

100 microliters of various compositions containing a surfactant, an oil, phosphate buffer (PBS) and 0.1 mg/cm³0f ovalbumin were injected subcutaneously into various batches of five female mice of the OF1 strain, weighing an average of 18 to 20 grams, at t=0 with a booster at t=28 days.

Blood samples are taken at 14, 28, 42, 56 and 90 days.

ELISA assays are carried out on the blood samples, for IgGIs in order to determine the humoral immune response, and IgG2as in order to determine the cellular immune response.

The compositions infected are as follows:

| Compositions | | | Ovalbumin |
|---|---|---|---|
| injected | Fatty phase | PBS | 0.1 mg/cm³ |
| Composition 1 | 1 ml containing 10% of | 1 ml | 20 μ |
| | composition D + 90% of | | |
| | MARCOL ™ 52 | | |
| Composition 2 | 1 ml containing 10% of | 1 ml | 20 μl |
| | composition E + 90% of | | |
| | MARCOL ™ 52 | | |
| Composition 3 | 1 ml containing 10% of | 1 ml | 20 μl |
| | composition F + 90% of | | |
| | MARCOL ™ 52 | | |
| Control | 1 ml of MARCOL ™ 52 | 1 ml | 20 μl |

The results of the ELISA assays are as follows:

IG1 assay (timescale in days)

| Composition | | | | | |
|---|---|---|---|---|---|
| injected | D14 | D28 | D42 | D56 | D90 |
| Composition | 30000 | 300000 | 1000000 | 700000 | 700000 |
| 1 | | | | | |
| Composition | 30000 | 300000 | 1000000 | 1000000 | 700000 |
| 2 | | | | | |
| Composition | 15000 | 100000 | 900000 | 600000 | 700000 |
| 3 | | | | | |
| Control | 0 | 0 | 80000 | 15000 | 15000 |

IG2 assay (timescale in days)

| Composition | | | | | |
|---|---|---|---|---|---|
| injected | D14 | D28 | D42 | D56 | D90 |
| Composition | <1000 | 2500 | 100000 | 30000 | 9000 |
| 1 | | | | | |
| Composition | <1000 | 6000 | 100000 | 50000 | 30000 |
| 2 | | | | | |
| Composition | <1000 | 6000 | 100000 | 50000 | 30000 |
| 3 | | | | | |
| Control | <1000 | <1000 | <1000 | <1000 | <1000 |

### EXAMPLE 2

100 microlitres of various compositions containing a surfactant, phosphate buffer (PBS) and 25 mg/cm³ of BSA were injected subCutaneously into various batches of five female mice of the OFI strain, weighing an average of 18 to 20 grams, at t=0 with a booster at t=28 days.

Blood samples are taken at 14. 28, 42, 56, 90 and 180 days.

ELISA assays are carried out on the blood samples, for IgGIs in order to determine the humoral immune response, and IgS2as in order to determine the cellular immune response The compositions injected are as follows:

| Composition | | | | | |
|---|---|---|---|---|---|
| injected | Adjuvant | PBS | | BSA | |
| Composition | 0.02 ml of a solution in PBS, | 1.8 | ml | 0.02 | ml |
| 4 | comprising 14.3% by weight of | | | | |
| | composition A. | | | | |
| Composition | 0.02 ml of a solution in PBS, | 1.98 | ml | 0.02 | ml |
| 5 | comprising 14.3% by weight of | | | | |
| | composition B. | | | | |
| Composition | 0.02 ml of solution in PBS, | 1.98 | ml | 0.02 | ml |
| 6 | comprising 20% by weight of | | | | |
| | composition C. | | | | |
| Composition | 2 ml of solution in PBS, | 1.8 | ml | 0.02 | ml |
| 7 | comprising 10% by weight of | | | | |
| | composition A. | | | | |
| Composition | 0.2 ml of a solution in PBS, | 1.8 | ml | 0.02 | ml |
| 8 | comprising 14.3% by weight of | | | | |
| | composition B. | | | | |
| Composition | 0.2 ml of a solution in PBS, | 1.8 | ml | 0.02 | ml |
| 9 | comprising 20% by weight of | | | | |
| | composition C. | | | | |
| Control | 0 | 2 | ml | 0.02 | ml |

The results of the ELISA assays are as follows:

IG1 assay (timescale in days)

| Composition | | | | | | |
|---|---|---|---|---|---|---|
| injected | D14 | D28 | D42 I | D56 | D90 | D180 |
| Composition | 1500 | 6000 | 126000 | 64000 | 8000 | 12000 |
| 4 | | | | | | |
| Composition | 8000 | 12000 | 128000 | 96000 | 12000 | 4000 |
| 7 | | | | | | |
| Composition | 1.000 | :000 | 32000 | 24000 | 3000 | 3000 |
| 5 | | | | | | |
| Composition | <1000 | 1500 | 64000 | 32000 | 12000 | 16000 |
| 8 | | | | | | |
| Composition | <1000 | <1000 | 12000 | 12000 | 4000 | <1000 |
| 6 | | | | | | |
| Composition | <1000 | 1500 | 24000 | 12000 | 3000 | <1000 |
| 9 | | | | | | |
| Control | <1000 | <1000 | 4000 | 6000 | <1000 | <1000 |

IG2 assay (timescale in days)

| Composition | | | | | | |
|---|---|---|---|---|---|---|
| injected | DI4 | D28 | D42 | D56 | D90 | D180 |
| Composition | <1000 | 2000 | 16000 | 8000 | 12000 | <1000 |
| 4 | | | | | | |
| Composition | <1000 | <1000 | 4000 | 3000 | 4000 | <1000 |
| 7 | | | | | | |
| Composition | <1000 | <1000 | <1000 | <1000 | <1000 | <1000 |
| 5 | | | | | | |
| Composition | <1000 | <1000 | <1000 | <1000 | <1000 | 16000 |
| 8 | | | | | | |
| Composition | <1000 | <1000 | 3000 | 3000 | 2000 | <1000 |
| 6 | | | | | | |
| Composition | <1000 | <1000 | 6000 | 6000 | 4000 | 2000 |
| 9 | | | | | | |
| Control | <1000 | <1000 | <1000 | <1000 | <1000 | <1000 |

The results set out above demonstrate the immunostimulant activity of the compounds of formula (I) alone, or in combination with the compounds of formula

What is claimed is:

1. A method of preventing or treating an infectious disease comprising administering a mixture comprising:
a) a compound of formula (I)
R₁—O-(6) ₅-H (I)
in which R₁ represents a saturated or unsaturated, linear or branched hydrocarbon-based radical comprising 1 to 30 carbon atoms, G represents the residue of a saccharine and x represents a decimal number between 1 and 5, or a mixture of compounds of formula (I) and/or
b) a compound of formula (II)
R₂—O (II)
in which R₂ represents, independently of R₁, a saturated or unsaturated, linear or branched hydrocarbon-based radical comprising from 8 to 30 carbon atoms, or a mixture of compounds of formula (II), as an adjuvant of immunity in an immunization composition comprising at least one antigen or at least one in vivo generator of a compound comprising an amino acid sequence.

2. The method according to claim 1, wherein in formula (I), the number x is between 1 and 3.

3. The method according to claim 1, wherein in formula (I), the number x is between 1.05 and 2.5.

4. The method according to claim 1, wherein in formula (I), the number x is between 1.1 and 2.0.

5. The method according to claim 1, wherein in formula (I), the number x is less than or equal to 1.5.

6. The method according to claim 5, according to which, in formula (I), G represents the residue of glucose or the residue of xylose.

7. The method according to claim 1, according to which, in formula (I), G represents the residue of glucose or the residue of xylose.

8. The method according to claim 1, according to which in formula (I), the R₁ radical represents a radical comprising from 5 to 22 carbon atoms, chosen from pentyl, hexyl, heptyl, octyl, nonyl, decyl. undecyl, dodecyl, tridecyl, tetradecyl, pentadecyl, hexadecyl, heptadecyl, octadecyl, nonadecyl, eicosyl, uneicosyl, docosyl, heptadecenyl, eicosenyl, uneicosenyl, docosenyl or heptadecadienyl or decenyl radicals, said radicals being linear or branched.

9. The method according to claim 1, according to which, in formula (I) R₁ represents a radical comprising from 8 to 20 carbon atoms, said radicals being linear or branched.

10. The method according to claim 1, according to which, in formula (I), R₂ represents a radical comprising from 8 to 22 carbon atoms, chosen from octyl, nonyl, decyl, undecyl, dodecyl, tridecyl, tetradecyl, pentadecyl. hexadccyl, heptadecyl, octadecyl, nonadecyl. eicosyl, uneicosyl, docosyl, heptadecenyl, eicosenyl, uneicosenyl, docosenyl or heptadecadienvl or decenyl radicals, said radicals being linear or branched.

11. The method according to claim 1, according to which, when the mixture comprises at least one compound of the formula (I) and at least one compound of formula (II), the compound of formula (I)/compound of formula (II) weight ratio is between 10/90 and 90/10, and more particularly between 10/90 and 60/40.

12. The method according to claim 1, an immunization composition comprising an aqueous phase and one or more oils chosen from mineral, animal or plant oils, in a weight proportion in oily phase of between 10 and 90% of the total weight thereof, and preferably between 30% and 70% by weight of the total weight thereof.

13. The method according to claim 1, in an immunization composition comprising an aqueous phase and not comprising an oily phase.

14. The method as defined in claim 1, for preparing a vaccine intended for preventing or for treating an infectious disease, in particular an infectious disease engendered by a virus or a micro-organism.

15. The method as defined in claim 1, for preparing a vaccine intended for preventing or treating a disease which is functional in nature, such as cancer or cystic fibrosis.

16. A composition comprising:
(i) at least one antigen or at least one in vivo generator of a compound comprising an amino acid sequence and,
(ii) at least one compound of formula (I):
R₁—O-(G) x-H (I)
in which R₁ represents a saturated or unsaturated, linear or branched hydrocarbon-based radical comprising 1 to 30 carbon atoms, G represents the residue of a saccharine and x represents a decimal number between 1 and 5, or a mixture of compounds of formula (I) and/or.

17. The composition of claim 16, further comprising:
(iii) at least one compound of formula (II), in which R₂ represents, independently of R₁, a saturated or unsaturated, linear or branched hydrocarbon-based radical comprising from 8 to 30 carbon atoms, or a mixture of compounds of formula (II), as an adjuvant of immunity in an immunization composition comprising at least one antigen or at least one in vivo generator of a compound comprising an amino acid sequence.

18. The composition as defined in claim 16, also comprising one or more oils chosen from mineral, animal or plant oils, in a weight proportion in oily phase of between 10 and 90% of the total weight thereof, and preferably between 30% and 70% by weight of the total weight thereof.

19. The composition an defined in claim 18, in the form of a water-in-oil (W/O) emulsion, of a water-in-oil (WIO/W) emulsion of a microemulsion.

20. The composition as defined in claim 16, not comprising an oily phase.

21. The composition as defined in claim 16, also comprising one or more water-soluble metal cation organic salts, such as for example calcium gluconate, manganese gluconate, manganese glycerophosohate, calcium glycerophosohate, calcium fructoheptonate, manganese fructoheptanate, aluminum salicylate or soluble aluminum acetate.

22. The composition as defined in claim 16, also comprising a sympathomimetic compound chosen from tyramine, isoproterenol, L-adrenaline. levarterenol, ephedrine, phenylephedrine or salbutamol.

23. The composition as defined in claim 16, for carrying out therapeutic-treatment of the human or animal body.

24. The composition as defined in claim 23, for carrying out therapeutic treatment of the human or animal body via the parenteral pathway, by subcutaneous, intramuscular or intraperitoneal injection.

25. The composition as defined in claim 23, for carrying out therapeutic treatment of the human or animal body via the mucosal pathway, in particular orally, rectally, nasally or vaginally.
